(19) United States
(12) Patent Application Publication
(10) Pub. No.: US 2006/0194299 A1
(43) Pub. Date: Aug. 31, 2006

(54) Method for recycling important nutritional elements from waste

(76) Inventors: Henrik Brinch-Pedersen, Skaelskor (DK); Sven Gjedde Sommer, Horsens (DK); Preben Bach Holm, Valby (DK)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/346,203

(22) Filed: Feb. 3, 2006

(30) Foreign Application Priority Data
Feb. 4, 2005 (DK) PA 2005 00178
Feb. 24, 2005 (DK) PA 2005 00282

(51) Int. Cl. C12N 9/00; C12N 9/10; C12N 9/14

(52) U.S. Cl. 435/183; 435/193; 435/195

(57) ABSTRACT

The present invention relates to a process wherein waste material derived from human, animal and industrial areas is processed to utilize the energy resources present in the solid phase and, optionally, to recover important nutritional elements as well as toxic heavy metals. In particular, there is provided a process for releasing plant nutritional elements and utilising toxic metals and carbon energy resources present in such waste, comprising treating the waste with one or more enzymes as biological catalysts.

### FIELD OF THE INVENTION

The present invention relates in general to the field of treatment of human, animal and industrial waste. In particular, there is provided a process for releasing plant nutritional elements and optionally combined with recovering toxic metals and utilising carbon energy resources present in such waste, comprising treating the waste with one or more enzymes which may be derived from microbes or plant tissues, or be purified enzyme preparations.

### TECHNICAL BACKGROUND OF THE INVENTION

There are increasing problems in the handling of human, animal and industrial waste material due the continuously increasing amount produced. The problems are global in nature, but are in particular acute in areas with very dense human populations and in areas with intense livestock production.

Spreading of sewage sludge and animal manures onto agricultural land has always been the simplest strategy for recycling plant nutrients, such as phosphate and nitrogen, contained in the sewage and manure. However, the livestock farms are often spreading a surplus of manure, and thus a surplus of plant nutrients, on the fields in proportion to the crop requirement. This results in a washing out of plant nutrients to aquifers and surface water, and thus they play a central role in the processes of eutrophication. In addition to the over-manuring, the risk of the washing out the nutrients is further increased as many of the plant nutritional elements in the waste are in a form which plants are not capable of absorbing. Furthermore, the presently handling of the manure may result in spreading of diseases. Obviously, in recent years, the whole practice of spreading has been called into question with pressure on heavy metal content, pathogens, odour, and the nutrient losses to water etc.

Thus, different solutions for removal of the nutritional elements from the manure have been proposed. One proposal is to store the manure for several months in order to decompose and transfer the nutrients, such as phosphate, from the solid phase of the manure to the liquid phase. Subsequently, the nutrients are recovered from the liquid phase by precipitation.

In contrast thereto, most of the sewage or waste from urban or industrial areas, is treated aerobically, anaerobically, chemically and/or mechanically producing, besides a liquid fraction of the waste, a dry matter solid fraction of the waste which is rich in energy and plant nutrients such as nitrogen and phosphate, but also contains toxic metals like arsenic, cupper, nickel, manganese, mercury and cadmium. The environmental aspects of using the solid waste directly as a fertilizer on agricultural land are thus under constant investigation and debate.

Due to the need of an environmental safe and hygienic handling of animal waste, there is focus on finding methods similar to those implemented for waste from urban and industrial areas. Presently, techniques have been developed ranging from a rather simple filter band and screw press separators to centrifugation and precipitation technology that efficiently separates the liquid phase from the solid phase of the animal waste material. However, the separation of manure has not yet really been implemented in the handling of manure.

Common for human, animal and industrial waste is that the solid waste fraction constitutes an important and valuable resource due to its content of different carbon compounds that may be converted to biogas or bioethanol, as well as important plant nutrients like phosphate and nitrogen that may be recycled or recovered. As mentioned above, many of the plant nutrients are in a non-soluble or non-available form, which makes them impossible to recycle as such, because plants are unable to absorb and utilise them. This results in turn in a multiplication of the amount of nutrients returned to the soil and water and in consequence an increased pollution of the aquatic environment.

For example, the recovery of phosphate from the solid fraction is in particular acute, because there is a very high phosphate load from the large agricultural areas with intensive livestock production. In seeds, the most common animal feed, typically 80% of the phosphate is bound as phytic acid (myo-inositol 1,2,3,4,5,6 hexakisphosphate). Phytic acid exists as a mixed salt and consists of myo-inositol with six phosphate molecules tightly binding a mixture of minerals such as Ca²⁺, K+ and Mg²⁺ but also Zn²⁺, Cu²⁺ and Fe²⁺. Non-ruminant animals, including human, are not capable of degrading phytic acid implying that most of the phytic acid is excreted, which is thus an environmental problem.

Furthermore, the shortage of bio-available phosphate in animal feeds is compensated by supplementation of typically mono- or di-calcium phosphate, also known as rock phosphate which unfortunately is a non-renewable resource. In order to utilize the phosphate resources in the feed the enzyme phytase is added to the feed as this enzyme degrades the phytate and makes the phosphate available for the animals.

However, there is a demand to find new resources for non-renewable nutrients for their use as feed additives but also for use as fertilizers on agricultural or horticultural land. Although waste and manure are known to contain such valuable resources, the industry within the field of waste treatment is not in the possession of any economically attractive method for releasing and recycling such important nutritional elements present in waste derived from human, animal or the industry.

Thus, there is an environmental and industrial need to find methods and processes for recovering, releasing and/or utilising the valuable nutrients in waste.

It is therefore one significant object of the present invention to provide a process for releasing and recycling important nutritional elements derived from waste. The inventors of the present invention found that there is a large potential of using various kinds of enzymes for solubilizing important nutritional elements present in the waste, such as manure, thereby facilitating release of energy and increase availability of important plant nutritional elements in the solid waste, but also for the safe removal of toxic heavy metals and pathogenic microorganisms. Furthermore, it was found that the separation of the waste into a liquid and solid fraction with low water content facilitates the above process.

The process of the invention has the advantages of being capable of 1) giving a very high degree of released valuable nutritional elements from waste including phosphate and nitrogen, 2) improving the utilisation of the carbon resources in the solid waste, 3) reducing the overall cost for the treatment of waste, 4) recovery of toxic metals and 5) eliminating potentially pathogenic microorganisms.

Thus, the process of the invention not only provides improved process economy, e.g. with respect of reduced cost for transportation of the waste, but also provide important nutrients for both plants and animal which are non-renewable in nature. Furthermore, the present invention provides a solution for avoiding the consequences for the environment of over-manuring and for an environmentally friendly removal of waste derived from human and the industry. Finally, it eliminates potential hazards such as toxic metals and pathogenic micro organisms.

### SUMMARY OF THE INVENTION

Accordingly, the present invention pertains to a process for releasing nutritional elements from waste, the process comprising the steps of: (a) separating the waste into a solid and a liquid phase; and (b) adding to said solid phase or a slurry prepared thereof at least one enzyme or at least one mixture of enzymes.

Thus, the starting point is a fraction of solid waste of human, animal or industrial origin derived from slurry or sewage separation and processing units. The solid waste may or may not have been subjected to fermentation for production of biogas or bioethanol. Furthermore, the separation of the waste should preferable produce a solid waste fraction that can be stirred and pumped through pipes. The solid waste is preferable heated and stirred and one enzyme or an enzyme cocktail is added simulatanously or sequentially to the solid waste.

The enzymes may belong to types that 1) can degrade cell wall materials such as xylanases, cellulases, glucanases or enzymes that can eliminate phenolic cross linking compounds in cell wall materials, 2) proteases that degrade residual proteins, 3) lipases that degrade lipids, 4) amylases that degrade starch, 5) ureases that degrade ureic acid and 6) phytases and phosphatases that degrade polyphosphate compounds like phytic acid. However, enzymes such as nucleases, glucosidases and esterases may also be useful in the present invention. The enzymes may originate from microbial fermentation that are added as particular formulations or they may be derived from microorganisms or plants that either due to native properties or as a result of genetic engineering produce enzymes of the types described above. Likewise, the enzymes may have different temperature and pH optima.

Following enzymatic treatments of the solid fraction of the waste, plant nutrients as well as toxic metals are concentrated by well known precipitation methods such as addition of anions or cations or through floc formation by addition of polyacrylamides (polymers), or via bio-sorption using waste product non-viable microbial cells.

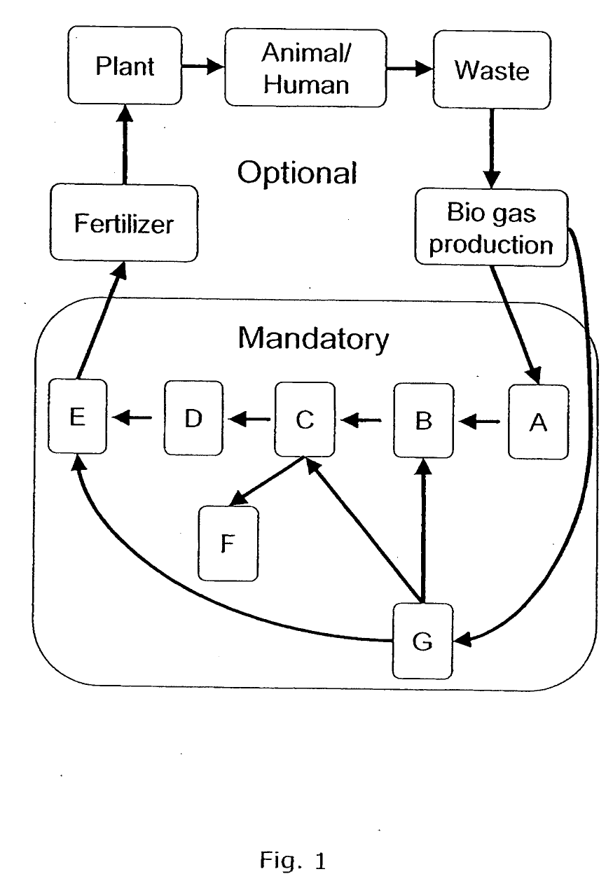

Thus, the present invention consists of a series of treatments, as shown in FIG. 1, where waste is separated in a liquid and a solid fraction (A). Slurry of the solid material is prepared in (B) and treated with enzymes in (C). After enzymatic treatments, nutritional elements, minerals and heavy metals are recovered in (D) and concentrated in (E). Carbohydrates are used for fermentation in (F) and excess energy (G) from biogas production is used during slurry preparation, enzymatic treatment, and during concentration of nutritional elements. The recovered nutrients re-enters the cycle as fertilizer in plant production giving rise to new animal feed and human food.

The present invention also pertains to the nutritional element obtained in the process according to the invention for use as an animal feed additive or as a fertiliser.

In a further aspect, the invention relates to the concentrate obtained in the process according to the present invention for use as an animal feed additive or a fertiliser.

In one useful aspect, the invention relates to an enzyme mixture comprising at least two enzymes, such as three, four, five, six, seven, eight, nine or ten enzymes, selected from xylanase, cellulase, hemicellulase, glucanase, urease, protease, lipase, amylase, phytase, phosphatase, aminopeptidase, amylase, carbohydrase, carboxypeptidase, catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, mannosidase, oxidase, pectinolytic enzyme, peptidoglutaminase, peroxidase, polyphenoloxidase, proteolytic enzyme, ribonuclease and transglutaminase, and the use of said enzyme mixture for releasing nutritional elements from waste.

### DETAILED DISCLOSURE OF THE INVENTION

Accordingly, the present invention has for its object to provide methods for enzyme mediated handling of solid waste derived from human, animal and industrial area, whereby the carbon resources are utilized as energy, the nutritional elements such as e.g. nitrogen and phosphate are recycled, heavy metals are removed and pathogenic microorganisms are eliminated.

The inventors of the present invention realised that it is possible to utilize the enormous resources present in waste and turning them into important and valuable nutritional elements. The essence of the invention lies in actively releasing nutritional elements, such as e.g. phosphate from phytic acid and/or nitrogen from nitrogen containing compounds, contained in the enriched solid fraction by subjecting the fraction to enzymatic treatments with cell wall, protein, lipid, starch and/or phytic acid degrading enzymes. This finding is also the basis for providing essential nutrients for plants and animals and relieves the environment of pollution problems due to the conventional discarded waste components.

Furthermore, the inventors realised that by separating the solid fraction of the waste from the liquid fraction a more effectively action of the enzymes can be obtained compared to the action of the enzymes in non-separated waste. This increased enzyme activity results in an increased release of nutritional elements and it has been shown a less amounts of enzymes are needed for efficient degradation. Furthermore, due to the separation of the waste, a solid fraction having a high concentration of dry matter and plant nutrients is obtained, which reduces the cost for transport to a central processing plant and a more efficient process control is possible by using the solid fraction.

One will realize that a complete separation of solid and liquid constituents of the waste is seldom feasible. Therefore, the solid phase may in addition to its content of dry matter comprise a certain amount of the original liquid. For example, the separation process may if not completely removing any original liquid, cause a reduction of the liquid content by 10%, 20%, 30%, 40%, 50%, 60%, 70%, 75%, 80%, 85%, 90%, 95%, 98% or 99%.

Thus, in an important aspect of the present invention, there is provided a process for releasing nutritional elements from waste, the process comprising the steps of (a) separating the waste into a solid and a liquid phase; and (b) adding to said solid phase or a slurry prepared thereof at least one enzyme or at least one mixture of enzymes.

In a presently preferred embodiment, the process according to the invention, comprising the steps of (i) separating the waste into a liquid and a solid phase, eventual with a consistence of the solids phase allowing it to be stirred or pumped through pipes; (ii) providing an aqueous slurry of said solid phase, (iii) adding to said slurry at least one enzyme or at least one mixture of enzymes; (iv) keeping the slurry of step (iii) under appropriate conditions resulting in at least partial release of the nutritional elements into said slurry, and (v) separating the nutritional elements resulting in step (iv) from said slurry.

It will be understood, that there, in the present context, is a difference between the liquid phase of the above step (i) and the slurry of step (ii) obtained by adding a liquid or water to the solid phase of the waste. For example, the water content in the liquid phase is at least 10% higher, such as at least 25% higher, including 50% higher compared to the water in the slurry.

In the present context, the expression “solid phase” is used interchangeable with the expression “solid fraction” and relates to the phase or fraction of the waste after the original water or liquid has been removed or partially removed, e.g. by a process selected from the group consisting of filtration, centrifugation, sedimentation and decanting. The separation of the two phases is as described later. In preferred embodiments, the solid phase contains after the separation from the liquid phase at the most 5% water or liquid, such as at the most 10%, e.g. at the most 15%, such at the most 20% including at the most 25% such as at the most 50%, e.g. at the most 60%, such at the most 75% including at the most 80% water or liquid.

In preferred embodiments, the nutritional elements or nutrients are selected from the group consisting of plant nutrients, metals, minerals and carbohydrates. In useful embodiments, the plant nutrients are selected from the group consisting of phosphate, calcium and nitrogen. In a preferred embodiment of the present invention, the plant nutrient is phosphate, such as organic phosphate or inorganic orthophosphate.

In further embodiments, the metals, such as heavy metals, which are removed in the process of the invention, are selected from the group consisting of arsenic, copper, nickel, manganese, mercury, cadmium, magnesium, zinc, cobalt, iron, molybdenum and boron. Such metals with the exception of cadmium, may be used as a nutrient compound in e.g. feed products or fermentation media.

In the present context, the terms “waste”, “sewage” and “refuse” are used interchangeable and refers to any type of discarded organic material derived from human, animal or industrial areas, which is non-separated and thus contain both a liquid and solid phase. In preferred embodiments, the waste is selected from the group consisting of municipal sewage, household waste, slaughterhouse waste, human waste, plant waste such as from gardening, animal waste and industrial waste such as waste from the food, feed and pharmaceutical industry, i.e. waste from fermentation processes, brewing or production of recombinant enzymes. The waste may be provided from waste holding facilities, i.e. facilities for holding, storage or treatment of waste, including pits or lagoon where animal waste preferable is stored.

A particular interesting embodiment of the present invention is where the animal waste is manure. As described above, manure constitutes an important resource, which, until now, has not been commercially exploited for the utilisation of valuable plant nutrients, i.e. nitrogen, phosphate and potassium, and carbohydrates. The content of nutrients and pH of the manure is mainly controlled by the animal species (Sommer and Husted, 1995).

The process of the invention, as illustrated in FIG. 1, involves the use of waste derived from human, animal or from the industry which optionally prior to the separation into a solid and a liquid phase, has been subjected to any kind of degradation to an initial release of nutrients and carbohydrates and/or to an aerobic or anaerobic fermentation process for production of biogas, bioethanol or any other kind of fermentation product. This is described later.

Subsequently, the solid phase is separated from the liquid phase. This may preferably be performed by centrifugation, filtration, sedimentation or decanting. It is well known that the solid phase of the waste contains desirable nutrients and carbohydrates. Under prolonged storage, such as 2-3 months, some of the nutrients would actively be transposed from the solid phase to the liquid phase, which is not desirable in the process according to the invention.

Thus, in order to obtain an as high a content of nutrients and carbohydrates as possible from waste, it is preferred to use the solid phase of fresh waste, i.e. waste which at the most has been stored for 2 months, such as at the most for 1 month, e.g. at the most 15 days, including at the most 10 days. However, it will be understood that under some circumstances it would be more practical to store the waste for a period of time, and subsequently precipitate and recover the nutrients which has been transposed from the solid phase to the liquid phase during storage, and treat the solid phase according to the invention.

In embodiments of the present invention where the animal waste is manure, it will be understood that separation or partial separation of the solid phase from the liquid phase will result in a reduction of the content of urine in the solid phase. In preferred embodiments of the invention the content of urine is reduced and constitutes at the most 35% of the wet weight of the solid phase, such as at the most 32,5%, 30%, 27,5%, 25%, 22,5%, 20%, 17,5%, 15%, 12,5%, 10%, 7,5%, 5%, or 2,5%. In the present context the term “urine” is used in its conventional meaning referring to the waste material that is secreted by the kidney in vertebrates, and is rich in end products of protein metabolism together with salts and pigments, and forms a clear amber and usually slightly acid fluid in mammals but is semisolid in birds and reptiles.

In a further step of the present process, water or liquid is added to the solid phase of the waste to obtain a slurry of the solid phase. By providing a slurry of the solid waste phase it is possible to pump the material. The provision of a slurry is preferable carried out at the waste management plant, i.e. the place where the slurry is further treated according to the invention. In the present process, the term “water” relates to any kind of aqueous liquid such as water from aquifers and surface water, but also whey or a buffer, e.g. a sodium acetate buffer, sodium citrate, is encompassed by the term.

Furthermore, by suspending the solid phase of the waste, a hydrolysis or separation or at least a partial separation of the solid phase material into fibre and nutritional elements occurs. For example, during such treatment the phytic acid, present in the solid phase, is dissolved in the aqueous solution. Thus, a preferred embodiment of the invention is wherein in step (b) or step (ii) an at least partial separation of the solid phase into fibre and nutritional elements occurs. The viscosity and solubility of the solid fraction material may be further improved by incubation at elevated temperatures.

The process temperature employed during the hydrolysis of the solid waste material is preferable between 0 and 82° C., such as between 15 and 55° C. In preferred embodiments, the temperature is at least 5° C., such as at least 15° C., e.g. at least 25° C. including at least 37° C., e.g. at least 40° C., such as at least 55° C. including at least 82° C. In further useful embodiments, the temperature in which step (b) or step (ii) of the process according to the invention is performed is less than 82° C., such as less than 55° C., e.g. less than 40° C. including less than 37° C., e.g. less than 25° C., such as less than 15° C. including less than 50° C.

It is, however, desired to set the temperature so as to obtain the desired separation of the waste material into fibre, i.e. cellulose, hemicellulose and lignin, and nutritional elements, without the destruction of to many nutritional elements, but also keeping polysaccharide molecules in tact, as these molecules serve as a direct nutrient for e.g. ethanol producing organisms in an optionally subsequent step of the present process.

Subsequently to the above hydrolysis of the solid waste material, the slurry is subjected to an enzymatic hydrolysis or degradation, which is achieved by treatment with one or more appropriate enzymes. For example, during the enzymatic hydrolysis the phosphate is released from the phytic acid. In preferred embodiments, two or more enzymes, such as three, four, five, six, seven, eight, nine or ten enzymes, are added to slurry of the solid phase. Under some circumstances it may be useful to add the two or more enzymes together or subsequently to the solid phase or slurry thereof.

In preferred embodiments, the enzyme is selected from the group consisting of xylanase, cellulase, hemicellulase, glucanase, urease, protease, lipase, amylase, phytase, phosphatase, aminopeptidase, amylase, carbohydrase, carboxypeptidase, catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, mannosidase, oxidase, pectinolytic enzyme, peptidoglutaminase, peroxidase, polyphenoloxidase, proteolytic enzyme, ribonuclease and transglutaminase, or combinations hereof. The addition of xylanase, glucanase and cellulose results in a degradation of the cell wall of the lignocellulosic material present in the waste, whereas the protease degrades protein, lipase degrades lipid and starch is degraded by the addition of amylase.

In useful embodiments, the enzyme is added to the slurry in a quantity of at least 1 ng per kg slurry dry weight, such as at least 5 ng per kg slurry dry weight, e.g. 10 ng per kg slurry dry weight, including at least 25 ng per kg slurry dry weight, such as at least 50 ng per kg slurry dry weight. In further embodiments, the enzyme is added to the slurry in a quantity of at least 1 μg per kg slurry dry weight, such as at least 5 μg per kg slurry dry weight, e.g. 10 μg per kg slurry dry weight, including at least 25 μg per kg slurry dry weight, such as at least 50 μg per kg slurry dry weight. In further useful embodiments, the enzyme is added to the slurry in a quantity of at least 1 mg per kg slurry dry weight, such as at least 5 mg per kg slurry dry weight, e.g. 10 mg per kg slurry dry weight, including at least 25 mg per kg slurry dry weight, such as at least 50 mg per kg slurry dry weight. In still further useful embodiments, the enzyme is added to the slurry in a quantity of at least 1 g per kg slurry dry weight, such as at least 5 g per kg slurry dry weight, e.g. 10 g per kg slurry dry weight, including at least 25 g per kg slurry dry weight, such as at least 50 g per kg slurry dry weight. In even further useful embodiments, the enzyme is added to the slurry in a quantity of at least 1 g per litre slurry, such as at least 1.5 g per litre slurry, e.g. 3 g per litre slurry, including at least 5 g per litre slurry, such as at least 10 g per litre slurry.

The amount of the enzyme added to the slurry is an amount which results in the presence in the slurry of 10 to 5000 units per litre slurry, such as in the range of 100 to 3000 units per litre slurry, including in the range of 250 to 2500 units per litre slurry, such as in the range of 500 to 1000 units per litre slurry, including in the range of 750 to 1000 units per litre slurry.

In useful embodiments, the enzyme is added to the slurry in a quantity of at such as 10 units per litre slurry, such as at least 20 units per litre slurry, including at least 30 units per litre slurry, such as at least 50 units per litre slurry, including at least 100 units per litre slurry, such as at least 200 units per litre slurry, including at least 300 units per litre slurry, such as at least 500 units per litre slurry. In further useful embodiments, the enzyme is added to the slurry in a quantity of at such as 1000 units per litre slurry, such as at least 1200 units per litre slurry, including at least 1300 units per litre slurry, such as at least 1500 units per litre slurry, including at least 100 units per litre slurry, such as at least 2000 units per litre slurry, including at least 3000 units per litre slurry, such as at least 5000 units per litre slurry.

The term “activity” when used in reference to an enzyme is a relative measure of the ability of the enzyme to react with a standard substrate at fixed standard conditions. Activity is measured in “units” which is defined as μmoles of substrate reacted per minute per gram of the measured sample at fixed standard conditions (herein after “a standard assay”). The activity is also a measure of the amount of active enzyme protein. An enzyme has a specific activity which is the activity of the pure enzyme protein in the standard assay. The specific activity is also measured in “units” which is defined as μmoles of substrate reacted per minute per gram of pure enzyme at fixed standard conditions. When the specific activity of an enzyme is known the amount of pure enzyme protein in a sample can be calculated. If a 1 g sample of a pure enzyme react with 100 μmoles of a substrate per minute in a standard assay, the specific activity of the enzyme is 100 Units per gram pure enzyme. If a 1 g sample of unknown enzyme activity reacts with 50 μmoles of a substrate per minute in the standard assay, the activity of the sample is 50 Units per gram and there is 0.5 g of pure enzyme protein in the sample.

In a particular useful embodiment, the enzyme is phytase. Phytase and phosphatase degrades phytate and makes phosphate available to human, animal and plants, but releases also minerals and amino acids bound in phytate. In useful embodiments, the phytase is added to the slurry in a quantity of at least 1 ng per kg slurry dry weight, such as at least 5 ng per kg slurry dry weight, e.g. 10 ng per kg slurry dry weight, including at least 25 ng per kg slurry dry weight, such as at least 50 ng per kg slurry dry weight. In further embodiments, the phytase is added to the slurry in a quantity of at least 1 μg per kg slurry dry weight, such as at least 5 μg per kg slurry dry weight, e.g. 10 μg per kg slurry dry weight, including at least 25 μg per kg slurry dry weight, such as at least 50 μg per kg slurry dry weight. In useful embodiments, the phytase is added to the slurry in a quantity of at least 1 mg per kg slurry dry weight, such as at least 5 mg per kg slurry dry weight, e.g. 10 mg per kg slurry dry weight, including at least 25 mg per kg slurry dry weight, such as at least 50 mg per kg slurry dry weight. In further useful embodiments, the phytase is added to the slurry in a quantity of at least 1 g per kg slurry dry weight, such as at least 5 g per kg slurry dry weight, e.g. 10 g per kg slurry dry weight, including at least 25 g per kg slurry dry weight, such as at least 50 g per kg slurry dry weight.

In useful embodiments, the phytase is added to the slurry in a quantity of at such as 10 units per litre slurry, such as at least 20 units per litre slurry, including at least 30 units per litre slurry, such as at least 50 units per litre slurry, including at least 100 units per litre slurry, such as at least 200 units per litre slurry, including at least 300 units per litre slurry, such as at least 500 units per litre slurry. In further useful embodiments, the phytase is added to the slurry in a quantity of at such as 1000 units per litre slurry, such as at least 1200 units per litre slurry, including at least 1300 units per litre slurry, such as at least 1500 units per litre slurry, including at least 100 units per litre slurry, such as at least 2000 units per litre slurry, including at least 3000 units per litre slurry, such as at least 5000 units per litre slurry. In further useful embodiments, the phytase is added to the slurry in an amount in the range of 10 to 5000 units per litre slurry, such as in the range of 100 to 3000 units per litre slurry, including in the range of 250 to 2500 units per litre slurry, such as in the range of 500 to 1000 units per litre slurry, including in the range of 750 to 1000 units per litre slurry.

In relation to the present invention it may be preferred to use one or more particular isoforms of each of the enzymes mentioned. More specifically, it may be feasible to select or develop isoforms, which have substrate specificities and activity profiles ideally suited for maximal activity in various types of waste.

As for phytase the naturally occurring phytase in wheat is a 6-phytase, the nomenclature referring to the position on phytate against which the enzymatic activity is directed. Phytases currently distributed for use in agriculture includes 3-phytases as well as 6-phytases. As phytate is used as a supplement in feed, focus has been on identifying or developing isoforms of the enzyme, which exert maximal activity in the digestive tract of either pigs or poultry. As an example, the various phytase isoforms have different pH-activity profiles. While the naturally occurring phytases from plants generally have a narrow optimum around pH 5.5, phytases manufactured for use in agriculture are active over a wider range of pH values. In the context of the present invention the use of enzymes with, for instance, a relative wide pH optimum may be preferred as this will reduce the requirements for exact control of process parameters during the enzymatic treatment of the solid phase. Similarly, it may be possible to identify particular isoforms of the enzymes, including particular isoforms of phytase, which have reduced sensitivity to inhibitors, such as metal ions, possibly present in the waste to be treated. In addition, isoforms of the enzymes may be selected which are dependent on co-factors that are abundant in the waste to be treated.

Depending on the nature of the waste it may be feasible to develop new isoforms of the enzymes with characteristics to match the particular application. Strategies for engineering of enzymes for improved activity under specific conditions are reviewed, for instance, in Tomschy et al., although with focus on phytase and the development of isozymes with improved activity at low pH.

If the enzymes are added together to the solid waste phase, the enzymes may preferable be added as a mixture or cocktail of enzymes or as a composition comprising multiple enzymatic activities. Such a mixture or composition can be a commercial product or be prepared at the waste management plant. In preferred embodiments, the mixture of enzymes comprises at least two enzymes, such as three, four, five, six, seven, eight, nine or ten enzymes, selected from the group consisting of xylanase, cellulase, hemicellulase, glucanase, urease, protease, lipase, amylase, phytase, phosphatase, aminopeptidase, amylase, carbohydrase, carboxypeptidase, catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, mannosidase, oxidase, pectinolytic enzyme, peptidoglutaminase, peroxidase, polyphenoloxidase, proteolytic enzyme, ribonuclease and transglutaminase, or combinations hereof. In the below examples, example of mixtures or compositions are described.

An useful embodiment of the invention is where the one or more enzymes, or the above mixture or composition of enzyme is added to the solid phase of the waste, i.e. before a liquid or water is added to the solid phase to obtain said slurry.

In one useful embodiment of the present invention, the enzymatic treatment is performed with an enzyme selected from the group consisting of an enzyme which originates from microbial fermentation, enzymes derived from a microorganism such as a genetic engineered microorganism and a plant, such as a genetic engineered plant.

Thus, in an useful embodiment of the present invention, the enzyme or polypeptide is produced by a method wherein a strain or a host cell is cultivated, said strain or said host cell is in its wild-type form capable of producing the polypeptide, and subsequently said polypeptide is recovered. A vector comprising a nucleotide sequence coding for the polypeptide may be introduced into said strain or said host cell using methods known in the art, such as by protoplast transformation, electroporation, conjugation, by Agrobacterium mediated transformation, transformation via particle bombardment or transformation via lipofection. The cells are cultivated in a nutrient medium suitable for production of the polypeptide using methods known in the art, including shake flask cultivation, small-scale or large-scale fermentation (including continuous, batch, fed-batch, or solid state fermentations) in laboratory or industrial fermentors performed in a suitable medium and under conditions allowing the polypeptide to be expressed and/or isolated. The resulting polypeptide may be recovered by methods known in the art. For example, the polypeptide may be recovered from the nutrient medium by conventional procedures including, but not limited to, centrifugation, filtration, extraction, spray-drying, evaporation, or precipitation.

The strain or the host cell may be a unicellular microorganism, e.g., a prokaryote, or a non-unicellular microorganism, e.g., a eukaryote. Useful unicellular cells are bacterial cells such as gram positive bacteria including, but not limited to, a Bacillus cell, or a Streptomyces cell, or gram negative bacteria such as E. coli and Pseudomonas sp. The host cell may be a eukaryote, such as a mammalian, insect, or fungal cell. In a preferred embodiment, the host cell is a fungal cell of a species of, but not limited to, Candida, Hansenula, Kluyveromyces, Pichia, Saccharomyces, Schizosaccharomyces, Yarrowia, Acremonium, Aspergillus, including A. niger, Fusarium, Humicola, Mucor, Myceliophthora, Neurospora, Penicillium, Thielavia, Tolypocladium, Trichoderma, Thermomyces, including T. lanuginosus or Rhizotonia, including R. solani.

It will be appreciated, that the strains or host cells may be selected from a genetically modified strain of one of the above microorganisms. As used herein the expression “genetically modified strain” is used in the conventional meaning of that term i.e. it refers to strains obtained by subjecting a microbial strain to any conventionally used mutagenization treatment including treatment with a chemical mutagen such as ethanemethane sulphonate (EMS) or N-methyl-N′-nitro-N-nitroguanidine (NTG), UV light or to spontaneously occurring mutants, including classical mutagenesis. Furthermore, it is possible to provide the genetically modified strain or cell by random mutagenesis or by selection of spontaneously occurring mutants, i.e. without the use of recombinant DNA-technology, it is envisaged that mutants of the above organisms can be provided by such technology including site-directed mutagenesis and PCR techniques and other in vitro or in vivo modifications of specific DNA sequences once such sequences have been identified and isolated.

There are many examples where plants are used as production vehicles for the production of particular proteins such as enzymes. For example, xylan, b-glucan and phytate degrading enzymes have been successfully produced in genetically recombinant plants. Such plants, most often genetically recombinant plants, may contain one or more exogenous gene sequences which encode one or more enzyme gene products. The gene product is expressed in recoverable quantities in the recombinant plants and can be isolated from the plants, if desired. In general, DNA sequences encoding enzymes having any of the above-described functionalities can be obtained from several microbial sources, including bacterial and fungal sources, as well as genes from higher organisms such as plants and animals. Cloning the gene or cDNA sequence of the desired enzyme can be achieved by several well-known methods. One method is to purify the enzyme of interest (or purchase a sample if commercially available) and determine its N-terminal amino acid sequence, as well as several internal amino acid sequences, using known methods. Oligonucleotide probes corresponding to the amino acid sequence are then constructed (again using known methods) and used to screen a genomic or cDNA library of the organism from which the enzyme was isolated. Positive hybrids are identified, characterized using known methods (restriction enzyme analysis, etc.), and cloned by known means to yield DNA fragments containing the coding sequence for the desired enzyme activity. (See, for instance, Current Protocols in Molecular Biology, Chapters 5 and 6.)

In accordance with the present invention, the slurry of step (iii) is kept under appropriate conditions resulting in at least partial release of the nutritional elements into said slurry. In the present context, the expression “appropriate conditions” relates to a specific temperature and pH which is suitable for the enzyme or enzymes used.

The process temperature, i.e. the temperature during the enzymatic hydrolysis, is preferable between 0 and 82° C., such as between 15 and 55° C. In preferred embodiments, the temperature is at least 5° C., such as at least 15° C., e.g. at least 25° C. including at least 37° C., e.g. at least 40° C., such as at least 55° C. including at least 82° C. In further useful embodiments, the temperature in which step (b) of the process according to the invention is performed is less than 82° C., such as less than 55° C., e.g. less than 40° C. including less than 37° C., e.g. less than 25° C., such as less than 15° C. including less than 5° C. However, the temperature employed should be the optimum temperature of the enzyme used in the process.

In many cases, the treatment performed in step (iii) may be carried out with satisfactory results without any adjustment of the pH, i.e. neutral, of the aqueous slurry before, or during, the performance of the treatment. However, for some types of waste materials it may be advantageous to adjust the pH of the waste material before obtaining the slurry and/or after the slurry is prepared. The pH may be decreased, i.e. acidic conditions, but in general the pH of the reaction mixture is increased (i.e. alkaline) by adding appropriate amounts of an alkali or base (e.g. an alkali metal hydroxide such as sodium or potassium hydroxide, an alkaline earth metal hydroxide such as calcium hydroxide, an alkali metal carbonate such as sodium or potassium carbonate or another base such as ammonia) and/or a buffer system. Thus, in an interesting embodiment of the present invention the aqueous slurry is subjected to alkaline conditions in step (ii). However, in a useful embodiment, the pH of the slurry during the process is below pH 8, such as below pH 7, e.g. below pH 6, including below pH 5.

It has been shown that by sustaining a constant movement of the suspension or slurry, the dissolution and degradations is improved. Thus, in a preferred embodiment, the slurry or suspension in step (b) or step (ii), or during all the steps of the present process is under a constant movement.

After the enzymatic treatments, the slurry of step (iv) contains valuable phosphate, nitrogen compounds, minerals and carbohydrates, which preferable can be recovered from the slurry for further use or recycling. However, in an interesting embodiment, the slurry in step (iv) is used as a liquid fertiliser applied directly to the soil of agricultural or horticultural areas or sprayed directly on leaves of growing plants.

In a preferred embodiment, the process according to the invention, comprises a further step (v) for recovering said nutritional elements, including phosphate and nitrogen compounds, from the slurry of step (iv).

In one preferred embodiment, the nutritional elements are recovered by means of precipitation ions added to the slurry of step (iv) resulting in a precipitation of said nutritional elements. The precipitation of the nutrients, such as phosphate, can be performed most efficient after lowering the temperature of the slurry to about 0-25° C., such as 4-10° C. Precipitating ions can be added, or already be present in the solution, such as potassium and ammonium in order to form potassium and ammonium taranakite (H₆(NH₄,K)₃Al(PO₄)₆, 18H₂O), brushite (CaHPO₄, 2H₂O) or struvite (Mg(NH₄,k)PO₄, 6H₂O). The major part of phosphate can be recovered from the liquid fraction by precipitation of struvite. Struvite can be formed by addition of magnesium oxide. In case the solution contains too little ammonia for struvite formation, extra ammonia can be added. Precipitation may also be performed by divalent ions such as Ca²⁺ or Mg²⁺ forming calcium phosphate and magnesium phosphate respectively or by other ions i.e. from FeCl₃, Al₂(SO₄)₃ or Fe₂(SO₄)₃. Similar principles may be applied to nitrogen containing compound that may be precipitated with e.g. ammonium binding substances such as zeolite, KAPTO etc.

In a useful embodiment, the precipitated nutritional elements are subsequently concentrated by means of an ion separator, a membrane system, electrodialysis or evaporation. When concentrate or precipitate is de-watered by e.g. drying or evaporation, a nutritional and mineral rich fertilizer is formed. The economic value can be further increased by adding N, P and K or by including the concentrate in feed as described below.

In an interesting embodiment of the invention, the nutritional elements from the slurry of step (iv) are recovered by concentrating said elements, i.e. by means of an ion separator, a membrane system, electrodialysis or evaporation.

In cases where large amounts of heavy metals are present in the aqueous solution of the waste, the heavy metals can be removed by a heat treatment and/or via microbial biosorption. Many types of yeast and other microbial genera are known to uptake or absorb metal species from dilute aqueous solutions, accumulating these inside or at the surface of the cell structure. The complexity of the microbial cell wall composition provides multiple cation binding sites. Therefore, metal ions uptake can result from several mechanisms, such as physical adsorption, ion exchange and coordination binding to functional groups at the surface of living and non-living cells. To keep the operation costs down, the development has focused on the use of industrial waste and non-living microorganisms as adsorbent materials for heavy metal bio-sorption. For example, non-viable cell from the brewing industry has been used with success.

Following recovery of the nutritional elements, the remaining liquid still contains large amounts of carbohydrates that can be used in fermentation processes for instance in the bio ethanol production. Carbohydrates such as microbial fermentable sugars can be utilized by one or more microorganisms to produce fermentation products such as ethanol. Any microorganism capable of converting glucose to ethanol can be used in the process according to the invention. For example, a suitable microorganism may be a mesophilic microorganism (i.e. one which grows optimally at a temperature in the range of 20-40° C.), e.g. a yeast also referred to as “baker's yeast”, Saccharomyces cerevisiae.

It will be understood, that a useful ethanol-fermenting organism can be selected from a genetically modified organism of one of the above useful organisms having, relative to the organism from which it is derived, an increased or improved ethanol-fermenting activity. The provision of genetically modified microorganisms is described above.

As described above, the solid waste, i.e. prior to providing an aqueous slurry of said solid phase, may be subjected to a (α) thermal treatment and/or (β) anaerobic or aerobic fermentation in order to produce a desirable fermentation product, such as methane.

Thus, in an interesting embodiment of the present invention, the solid phase is burned in order to release nutrients. The ashes containing high concentration of solid phase nutrients is suspended, treated with at least one of the enzymes, in particular of interest phytase, and precipitated as already described for the solid fraction.

An anaerobic or aerobic fermentation my employ one or more fermenting microorganisms capable of degrading or converting substances present in the waste, i.e. liquid and solid phase, to form e.g. combustible fuel such as methane. In one useful embodiment of the present invention, an initial treatment of the waste is performed using methane-producing microorganisms (also known as methanogens), which constitute a group of prokaryotes that are capable of forming methane from certain classes of organic substrates, methyl substrates or acetate under anaerobic conditions. It will be appreciated that useful methanogenic bacteria can be selected from a genetically modified bacterium of known methanogenic bacteria, having, relative to the organism from which it is derived, an increased or improved methane producing activity. Other useful microorganisms which could be used in an anaerobic fermentation of the waste include certain fermentative anaerobic bacteria capable of converting, for example, glucose to products such as acetate, propionate, butyrate, hydrogen and CO₂, and so-called acetogenic bacteria, which convert organic substances such as propionate, butyrate and ethanol to acetate, formate, hydrogen and CO₂.

The process according to the invention is in particular suitable for applications coherent with large-scale waste management systems (see example in FIG. 1). When the process is performed downstream biogas production, excess heat from the biogas production plant can be utilized as process energy in the incubation processes.

It should be understood that any embodiments and/or feature discussed above in connection with the process according to the invention apply by analogy to the below aspects of the present invention.

In a further aspect, the present invention provides the nutritional element obtained in the process according to the invention for use as an animal feed additive or as a fertiliser.

In further aspects, the invention relates to the concentrate obtained in the process according to the invention for use as an animal feed additive or a fertiliser.

In yet another aspect, the present invention provides an enzyme mixture comprising at least two enzymes, such as three, four, five, six, seven, eight, nine or ten enzymes, selected from the group consisting of xylanase, cellulase, hemicellulase, glucanase, urease, protease, lipase, amylase, phytase, phosphatase, aminopeptidase, amylase, carbohydrase, carboxypeptidase, catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, mannosidase, oxidase, pectinolytic enzyme, peptidoglutaminase, peroxidase, polyphenoloxidase, proteolytic enzyme, ribonuclease and transglutaminase, or combinations hereof.

In a still further aspect, the invention relates to the use of the enzyme mixture according to the invention for releasing nutritional elements from waste.

The following examples are included to demonstrate particular embodiments of the invention. However, those of skill in the art should, in view of the present disclosure, appreciate that many changes can be made in the specific embodiments which are disclosed and still obtain a like or similar result without departing from the spirit and scope of the invention. The following examples and figures are offered by way of illustration and are not intended to limit the invention in any way, wherein

FIG. 1 illustrates the series of treatments, divided in a mandatory and an optional phase. Mandatory: A: separation of waste; B: providing a slurry of the solid phase; C: enzymatic treatments; D: recovery of nutritional elements, minerals, heavy metals; E: concentrating nutritional elements; F: fermentation of carbohydrates; G: excess energy. Optional: the recovered nutrients re-enters the cycle as fertilizer in plant production giving rise to new animal feed and human food; and

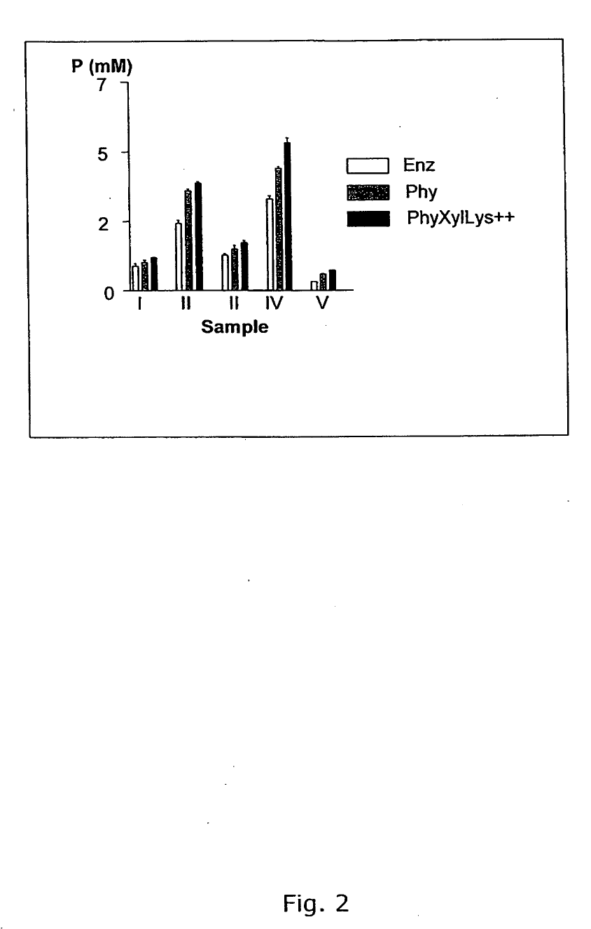

FIG. 2 shows the inorganic orthophosphate after 1 hr of incubation with no enzymes, with phytase, or with a cocktail consisting of phytase, xylanase and lysing enzymes.

### EXAMPLES

### Example 1

### Process for Releasing Nutritional Elements from Pig Manure

Manure from pigs is separated and 100 g of the solid fraction is suspended in 1 litre of water at room temperature and during constant stirring. After 2 hrs, an enzyme cocktail is added and the suspension is incubated for additional 8 hrs at 40° C. under constant stirring (100 rpm) of the suspension. The enzyme cocktail is composed of phytase (10,000 units/kg), beta-glucanase (35,000 units/kg), cellulase (11,000 units/kg), xylanase (400,000 units/kg), protease (0.25%), lipase (0.1%) and amylase (3,300,000 units/kg). After total of 10 hrs, 0.75% magnesium oxide and 0.3% NH₃ is added to the suspension for struvite formation, the stirring is stopped and the incubation is continued for another 2 hrs. Thereafter another 0.3% magnesium oxide and 0.4% NH₃ is added and the suspension is left for an additional 8 hrs where after the suspension is filtered and the liquid is stored to be used in fermentation processes. At this stage, the PO₄ content in the liquid is reduced by approximately 80% as phosphate is left as precipitate consisting mainly of struvite.

### Example 2

### Process for Releasing Nutrients from Different Waste Materials

The effect of the addition of the enzyme phytase or an enzyme cocktail of phytase, xylanase and lysing was tested in different waste materials, including in non-separated waste material and in the solid fraction of such waste materials.

2.1 Materials and Methods

The experiments included five waste materials:
(I) untreated pig manure from fatteners, i.e. containing both a solid and a liquid fraction;
(II) solid fraction produced by separating the pig manure from fatteners (I) with a decanter centrifuge;
(III) untreated pig manure from fatteners, i.e. containing both a solid and a liquid fraction;
(IV) solid fraction produced by separating the pig manure from fatteners (III) and adding flocculants and coagulants to the manure combined with dewatering in a belt press band separator;
(V) slaughterhouse meat waste with a consistence similar to minced pork.

From each waste material, 5.0 g sample portions were weighed in duplicate and resuspended in 250 ml 220 mM sodium-acetate buffer (pH 5.5). Each sample portions were either treated with a treatment designated a), b) or c) wherein:
Phy⁺: Aspergillus niger phytase (EC 3.1.3.8) (Sigma P9792) was added to a final concentration of 500 units/L;
PhyXylLyt⁺⁺⁺: the following enzyme cocktail was added: Aspergillus niger phytase to a final concentration of 500 units/L, xylanase from Thermomyces lanuginosus (Sigma X2753) to a final concentration of 3 g/L and lysing enzymes from Rhizotonia solani to a final concentration of 1.5 g/l. The lysing enzyme contained glucanases, proteases and cell lytic activities as described by the manufacturer (Sigma L8757);
Enz⁻: No enzymes were added.

The sample suspensions were incubated for 1 hour at 37° C., under constant vortexing (60× rpm). After incubation, the samples were centrifuged at 6000× g, 4° C. for 10 min and the supernatant was isolated. Two ml of the supernatant was mixed with 4.0 ml colour-stop mix, a standard ammonium heptamolybdate-ammonium vanadate solution as described in detail by Engelen et al. 1994. After adding the colour stop mix samples were centrifuged at 10000× g for 5 min and the absorbance was measured at 415 nm with a spectrophotometer (Helioβ, Unicam). The inorganic orthophosphate concentration was determined using a standard curve.

Samples I and II were selected for demonstrating the effect of enzyme additions on the release of copper and zinc from the non-separated manure and the solid phase of the manure. After incubation and centrifugation, 5 ml supernatant from each of I-PhyXylLyt⁺⁺⁺, I-Enz⁻, II-PhyXylLyt⁺⁺⁺ and II-Enz⁻ was isolated and analysed via ICP, using a standard protocol. The Ions were determined using ICP and each result (Table 2.1.) represents an average of two repeats.

2.2 Results

Results on the inorganic orthophosphate concentrations after incubations are presented in FIG. 2. In the two untreated or non-separated waste samples I and III, phytase activity during the incubation resulted in an orthophosphate content on 10.3 mM and 15.1 mM respectively, a 15% and 22% increase compared to the 8.9 mM and 12.8 mM in the corresponding samples where no enzymes were included. Addition of the enzyme cocktail of phytase, xylanase and lysing enzymes increased the orthophosphate content further to 11.7 mM and 17.4 mM for samples I and III respectively, a total increase of 31 and 36% compared to the samples where no enzymes were added.

In the solid waste fractions II and IV, the orthophosphate concentrations after incubation with phytase were 36 mM and 44 mM respectively in comparison to 24 mM and 33 mM in the corresponding samples where no enzyme were included during incubation. Thus, incubation with phytase caused a 48% and 33% higher orthophosphate concentration, respectively, than in the corresponding samples where no enzyme were added to the incubation. Incubation with the enzyme cocktail of phytase, xylanase and lysing enzymes increased the concentrations of orthophosphate further to 39 mM and 53 mM for samples II and IV, respectively, a 60% and 61% higher orthophosphate content than obtained when no enzymes were included during the incubation.

In the slaughterhouse waste samples V, the concentration of orthophosphate after incubation with no enzymes was 3.15 mM. Inclusion of phytase increased the concentration by 87% to 5.9 mM, whereas the enzyme cocktail of phytase, xylanase and lysing enzymes increased the concentration to 6.9 mM, a 219% increase compared to samples where no enzymes were included during the incubation.

The contents of copper and zinc ions after incubations are presented in Table 2.1. Without enzyme additions, the copper concentrations were below the detection limit of 0.02 mg/l. Similarly when no enzymes were added to sample I, the concentration was below the detection level of 0.05 mg/l. However, for both zinc and copper, the ion concentrations increased significantly when enzymes were included during the incubation. In sample I the copper content increased to 0.12 mg/l, whereas in sample II a copper content of 0.59 mg/l was measured. With regard to the content of zinc in sample I, the enzyme cocktail increased the concentration to 0.21 mg/l, whereas in sample II the concentration was 1.7 mg/l when the enzyme cocktail was included during incubation. Thus, a 274% increase compared to sample II (0.62 mg/l) was obtained.

TABLE 2.1
Concentration of free ions of copper and zinc in the supernatant of samples
I and II after incubation with and without the PhyXylGlu enzyme cocktail

| | I | I | II | II |
|---|---|---|---|---|
| | Enz⁻ | PhyXylGlu⁺⁺⁺ | Enz⁻ | PhyXylGlu⁺⁺⁺ |
| Copper (mg/l) | <0,02 | 0.12 | <0.02 | 0.59 |
| Zinc (mg/l) | <0.05 | 0.21 | 0.62 | 1.7 |

2.3 Conclusions

The above results clearly demonstrate an effect of the addition of one or more enzymes to waste materials. This effect is especially significant when the one or more enzymes are added to the solid phase of the waste compare to untreated or non-separated waste. Thus, an increased content of orthophosphate was observed when treating the waste with phytase or a cocktail of different enzymes.

When comparing the above results obtained from samples I and III, i.e. untreated or non-separated waste, with the samples II and IV, i.e. solid fraction of the waste, it is clear that there is an effect of the separation of the waste in a solid and liquid waste.

In the two solid waste samples, phytase activity during the incubation resulted in an average of 40.5% increased orthophosphate content compared to the content seen in the absence of phytase activity. In the non-separated waste samples the average increase in the orthophosphate content was a modest 18.5%.

The same results were seen for the use of enzyme cocktails. In the two solid waste samples, enzyme activity during the incubation resulted in an average of 60.5% increased orthophosphate content compared to the content seen in the absence of enzyme activity. In the non-separated waste samples the average increase in the orthophosphate content was a modest 33.5%.

Furthermore, the results show that even more nutrients can be released when a cocktail of different enzymes are used compared when only using a single enzymes such as phytase.

With regard to zinc and copper, the ion concentrations increased significantly when one or more enzymes were included during the incubation. Again there was seen a significant effect of the separation of the waste, as the ion concentration was relatively higher in the solid waste that in the non-separated waste.

From the above results it can be concluded that the process described herein is an effective way for releasing the nutritional elements, such as e.g. phosphate from phytic acid and ions, contained in the enriched solid fraction of the waste. These results are the basis for providing essential nutrients for plants and animals and relieves the environment of pollution problems due to the conventional discarded waste components.

### REFERENCES

Engelen, A J, Vanderheeft F C, Randsdorp P H G and Smit E L C 1994 Simple and Rapid-Determination of Phytase Activity. Journal of Aoac International 77, 760-764.Marques,

P. A. S. S., Rosa, M. F., and Pinheiro, H. M. 2000. pH effects on the removal of CU2⁺, Cd2⁺ and Pb2⁺ from aqueous solution by waste brewery biomass. Bioprocess Engineering 23:135-141.

Sommer, S. G. and Husted, S. 1995. The chemical buffer system in raw and digested animal slurry. Journal of Agricultural Science 124, 45-53.

Tomschy, A., Brugger, R., Lehmann, M., Svendsen, A., Vogel, K., Kostrewa, D., Lassen, S. F., Burger, D., Kronenberger, A., van Loon, A. P. G. M., Pasamontes, L. and Wyss, M. 2002. Engineering of Phytase for Improved Activity at Low pH Applied and Environmental Microbiology, 68:1907-1913.

1. A process for releasing nutritional elements from waste, the process comprising the steps of:
(a) separating the waste into a solid phase and a liquid phase; and
(b) adding to said solid phase or a slurry prepared from the solid phase at least one enzyme or at least one mixture of enzymes.

2. The process according to claim 1, comprising the steps of:
(i) separating the waste into a solid and a liquid phase;
(ii) providing an aqueous slurry of said solid phase,
(iii) adding to said slurry at least one enzyme or at least one mixture of enzymes;
(iv) keeping the slurry of step (iii) under appropriate conditions resulting in at least partial release of the nutritional elements into said slurry.

3. The process according to claim 1, wherein the nutritional elements are selected from the group consisting of plant nutrients, metals, minerals and carbohydrates.

4. The process according to claim 3, wherein the plant nutrients are selected from the group consisting of phosphate, calcium, nitrogen, and a mixture thereof.

5. The process according to claim 4, wherein the plant nutrient is at least one phosphate.

6. The process according to claim 3, wherein the metals are selected from the group consisting of arsenic, copper, nickel, manganese, mercury, cadmium, magnesium, zinc, cobalt, iron, molybdenum, boron and a mixture thereof.

7. The process according to claim 1, wherein the waste is selected from the group consisting of municipal sewage, household waste, slaughterhouse waste, human waste, animal waste industrial waste and a mixture thereof.

8. The process according to claim 7, wherein the animal waste is manure.

9. The process according to claim 1, wherein the solid phase of the waste is separated from the liquid phase by centrifugation or filtration.

10. The process according to claim 1, wherein water is added to the solid phase in step (b) of claim 1.

11. The process according to claim 1, wherein in step (b) at least partial separation of the solid phase into fibre and nutritional elements occurs.

12. The process according to claim 1, wherein two or more enzymes, three, four, five, six, seven, eight, nine or ten enzymes, are added to the solid phase or the slurry.

13. The process according to claim 12, wherein the two or more enzymes are added together or sequentially to the solid phase or the slurry.

14. The process according to claim 1, wherein the enzyme is selected from the group consisting of xylanase, cellulase, hemicellulase, glucanase, urease, protease, lipase, amylase, phytase, phosphatase, aminopeptidase, amylase, carbohydrase, carboxypeptidase, catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, mannosidase, oxidase, pectinolytic enzyme, peptidoglutaminase, peroxidase, polyphenoloxidase, proteolytic enzyme, ribonuclease and transglutaminase.

15. The process according to claim 1, wherein the mixture of enzymes comprises at least two enzymes, such as three, four, five, six, seven, eight, nine or ten enzymes.

16. The process according to claim 14, wherein the enzyme is phytase.

17. The process according to claim 16, wherein the phytase is added to the slurry in a quantity of at least 1 ng per kg slurry dry weight.

18. The process according to claim 1, wherein the enzymes are selected from the group consisting of enzymes originating from microbial fermentation, enzymes derived from microorganism, and enzymes derived from plants.

19. The process according to claim 1, wherein the slurry in step (ii) is under a constant movement.

20. The process according to claim 1, wherein the pH of the slurry during the process is below pH 8.

21. The process according to claim 2, wherein the slurry in step (iv) is used as a liquid fertiliser.

22. The process according to claim 2, wherein the process comprises a further step (v) for removing said nutritional elements from the slurry of step (iv).

23. The process according to claim 22, wherein the nutritional elements are recovered by means of precipitation ions added to the slurry of step (iv) resulting in a precipitation of said nutritional elements.

24. The process according to claim 23, wherein the precipitation ions are selected from the group consisting of potassium, ammonium, Ca²⁺, Mg²⁺, FeCl₃, Al₂(SO₄)₃ and Fe₂(SO₄)₃.

25. The process according to claim 23, wherein the precipitation ions are added to said slurry at a low temperature.

26. The process according to claim 22, wherein the nutritional elements are concentrated by means of an ion separator, a membrane system, electrodialysis or evaporation.

27. The process according to claim 22, wherein the nutritional elements from the slurry are recovered by concentrating said nutritional elements.

28. The process according to claim 27, wherein the nutritional elements are concentrated by means of an ion separator, a membrane system, electrodialysis or evaporation.

29. The process according to claim 1, wherein prior to providing an aqueous slurry of the solid phase of the waste, the solid waste is subjected to any of the following steps in any given order: thermal treatment, anaerobic, fermentation, aerobic fermentation, or any a combination thereof.

30. A method of modifying animal feed comprising adding to the animal feed the nutritional elements released by the process according to claim 1.

31. A method of modifying an animal feed, comprising adding to the animal feed the concentrate obtained in the process according to claim 26.

32. An enzyme mixture comprising at least two enzymes, three, four, five, six, seven, eight, nine or ten enzymes,

33. A method for releasing nutritional elements from waste comprising adding to the waste the enzyme mixture according to claim 32.

34. The process according to claim 2, wherein the slurry in step (b) is under a constant movement.

35. The process according to claim 2, wherein in step (ii) at least partial separation of the solid phase into fibre and nutritional elements occurs.

36. The process according to claim 1, wherein the mixture of enzymes comprises at least two or more of xylanase, cellulase, hemicellulase, glucanase, urease, protease, lipase, amylase, phytase, phosphatase, aminopeptidase, amylase, carbohydrase, carboxypeptidase, catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, mannosidase, oxidase, pectinolytic enzyme, peptidoglutaminase, peroxidase, polyphenoloxidase, proteolytic enzyme, ribonuclease or transglutaminase.

37. An enzyme mixture according to claim 32 comprising at least two or more of xylanase, cellulase, hemicellulase, glucanase, urease, protease, lipase, amylase, phytase, phosphatase, aminopeptidase, amylase, carbohydrase, carboxypeptidase, catalase, chitinase, cutinase, cyclodextrin glycosyltransferase, deoxyribonuclease, esterase, alpha galactosidase, beta-galactosidase, glucoamylase, alpha-glucosidase, beta-glucosidase, haloperoxidase, invertase, laccase, mannosidase, oxidase, pectinolytic enzyme, peptidoglutaminase, peroxidase, polyphenoloxidase, proteolytic enzyme, ribonuclease or transglutaminase.

38. The process according to claim 1, wherein the enzymes are selected from the group consisting of enzymes originating from enzymes derived from genetic engineered microorganisms and genetic engineered plants.

39. A method of enhancing nutrition of vegetation comprising adding to the soil in need thereof the nutritional elements released by the process according to claim 1.

40. A method of enhancing nutrition of vegetation comprising adding to the soil of agricultural or horticultural areas or spraying on leaves of growing plants the slurry of step (iv) according to claim 2.

41. A method of enhancing nutrition of vegetation comprising adding to the soil in need thereof the concentrate obtained in the process according to claim 26.

42. An enzyme mixture comprising at least two or more of xylanase, cellulase, hemicellulase, glucanase, urease, protease, lipase, amylase, phytase, phosphatase, aminopeptidase, amylase, carbohydrase, carboxypeptidase, catalase, chitinase, cutinase, cyclodextrin glycosyltransferase, deoxyribonuclease, esterase, alpha galactosidase, beta-galactosidase, glucoamylase, alpha-glucosidase, beta-glucosidase, haloperoxidase, invertase, laccase, mannosidase, oxidase, pectinolytic enzyme, peptidoglutaminase, peroxidase, polyphenoloxidase, proteolytic enzyme, ribonuclease or transglutaminase.

43. A method for releasing nutritional elements from waste comprising adding to the waste the enzyme mixture according to claim 37.
